(19) United States
(12) Patent Application Publication
(10) Pub. No.: US 2012/0130209 A1
(43) Pub. Date: May 24, 2012

(54) Implantable Sensor Unit

(75) Inventor: Andreas Bunge, Leipzig (DE)

(73) Assignee: BIOTRONIK SE & CO. KG, Berlin (DE)

(21) Appl. No.: 13/294,626

(22) Filed: Nov. 11, 2011

Related U.S. Application Data
(60) Provisional application No. 61/416,774, filed on Nov. 24, 2010.

(51) Int. Cl. A61B 5/1459; B82Y 15/00

(52) U.S. Cl. 600/316; 600/317; 977/774

(57) ABSTRACT

A sensor unit functionally stable in an implant, for performing qualitative and/or quantitative in vivo determination of an analyte, including a connection area (3) having at least one binding site for the analyte (7), the connection area undergoing a spatial change when the analyte binds to the binding site. The sensor unit further includes first (1) and second (5/9) fluorescently active regions bound to the connection area (3) in a manner such that, when a spatial change occurs to the connection area (3) due to the analyte binding to the connection area (3), the distance between the first fluorescently active region (1) and the second fluorescently active region (5/9) changes without the bonds of the fluorescently active regions (1, 5/9) to the connection area (3) being broken, and wherein one or both of the fluorescently active regions (1, 5/9) includes more than 60% by weight of an anorganic material.

### RELATED APPLICATION

This patent application claims the benefit of co-pending U.S. Provisional Patent Application No. 61/416,774, filed on Nov. 24, 2010, which is hereby incorporated by reference in its entirety.

### TECHNICAL FIELD

The invention relates to a sensor unit that is functionally stable in the implant, for performing the qualitative and/or quantitative in vivo determination of an analyte, an implant comprising such a sensor unit, a method for the qualitative and/or quantitative determination of an analyte in the body of a human or an animal, and the use of such a sensor unit and/or a related implant for the qualitative and/or quantitative determination of an analyte.

### BACKGROUND

The mode of operation of the human and animal body is based on a large number of biochemical processes that can be described in a qualitative and/or quantitative manner based on the presence of certain compounds in the body. For example, glucose concentration provides a great deal of information about the metabolic state of the particular body. The metabolic and anabolic processes in the body are based on a large number of balanced equilibrium states that communicate with each other. Metabolic disorders cause a portion of these equilibrium states to become disrupted. In many cases, certain active agents can be administered for therapy to correct the unwanted concentrations of certain compounds in the body, which are caused by metabolic disorders (or other diseases), by restoring metabolic equilibrium. The most commonly known and most economically relevant example of this situation is glucose concentration in the blood and/or plasma in persons with diabetes. Unwanted glucose levels can be restored to desired levels by administering the correct dosage of insulin. The dosage of insulin to be administered is dependent, of course, on the glucose concentration to be changed. It is therefore desirable to be able to obtain information about the particular glucose level in real time if possible. The same applies for compounds (analytes) other than glucose, such as, for example, hormones, electrolytes, lipids, amino acids, peptides, proteins, neurotransmitters, and end products of metabolism.

Various methods for performing the continuous, optical, in vitro determination of analyte concentrations are described in the literature. A primary example of such literature is the measurement of fluorescence.

One method for determining the concentration of glucose, per Pickup et al., In Vivo Glucose Monitoring: The Clinical Reality and the Promise, Biosensors and Bioelectronics 20 (2005) 1897-1902, is based on a displacement reaction. This method uses the protein concanavalin A (“Con A”) which has four binding sites for glucose under physiological conditions as a tetramer. Con A is bonded covalently with an organic fluorescent dye (e.g., APC). In the glucose-free state, fluorescence-labeled dextran molecules (fluorescent polymers comprised of glucose and fluorophor) are bound to the four binding sites. The association of the fluorescence-labeled dextran molecules with fluorescent Con A can be detected using, for example, fluorescence resonance energy transfer (“FRET”). FRET is based on energy transfer from a donor fluorophore to an adjacent, fluorescent acceptor and can be detected via a decrease in the donor fluorescence and/or an increase in the acceptor fluorescence. If glucose is present in the sample solution, the glucose displaces the bound, labeled dextran molecules. As a result, fewer fluorescent dextran molecules are located in the vicinity of Con A, which is observed as a reduction of the FRET.

Another method, according to Ye et al., Genetic Engineering of an Allosterically Based Glucose Indicator Protein for Continuous Glucose Monitoring by Fluorescence Resonance Energy Transfer, Anal. Chem. 2003, 75, 3451-3459, for determining glucose concentration is based on the conformational change in the glucose binding protein (“GBP”) which performs important functions in bacteria. In terms of structure, GBP is a type of hinge, to the pivot point of which glucose can bind selectively. The binding of the glucose induces the conformational change of the GBP. This is also detected using, for example, FRET. For this purpose, fluorescent molecules in the form of special proteins are bound to different positions of the GBP molecule. In the glucose-free state, the compact, three-dimensional structure of the GBP causes the two fluorescent organic molecular parts to be separated by a short distance: i.e., the FRET is great. If glucose is present in the sample solution, the glucose binds to the GBP, and a reduced FRET is observed due to the conformational change of the GBP and the resultant greater separation between the fluorescent proteins. The FRET changes as a function of glucose concentration, up to the concentration at which all GBPs are saturated with glucose.

As an alternative, a method is described in the literature (Medintz et al., Self-Assembled Nanoscale Biosensors Based on Quantum Dot FRET Donors, Nature Materials Vol. 2, September 2003, pages 630-638) for determining the concentration of a disaccharide on the basis of anorganic, fluorescent nanoparticles which are referred to as quantum dots (“QDs”). QDs have the following interesting properties compared to the most commonly used organic fluorescent dyes, such as, for example, rhodamine, NBD, or special proteins: QDs are characterized by high photostability and relatively great brightness. Their excellent optical properties can be adapted very easily for the required application by adjusting their size during synthesis. The detection of the disaccharide maltose is based on a displacement reaction. The maltose binding protein (“MBP”) is the detection element in this case. This molecule cannot bind a monosaccharide (glucose), however, due to its high selectivity for maltose. For optical detection, a quantum dot is bound to the MBP. The methodology requires an additional reagent, for example, a cyclodextrin (cyclic sugar molecule) with a covalently bound fluorescence quencher (QSY-9). In the absence of maltose, the cyclodextrin is bound to the MBP, and the fluorescence quencher quenches the fluorescence of the quantum dot. If maltose is present in the sample solution, the maltose displaces the cyclodextrin, and the fluorescence is quenched to a lesser extent and increases. The concentration of maltose can be deduced by detecting the fluorescence which occurs as a function of the maltose concentration.

The literature also contains discussions of a detection system for calcium (Ca²⁺) (Miyawaki et al., Fluorescent Indicator for Ca²⁺ Based on Green Fluorescent Proteins and Calmodulin, Nature Vol. 388, Aug. 28, 1997, pages 882-887). The focus thereof is on selectively Ca²⁺-binding calmodulin which is modified in two positions with fluorescent proteins. This construct is used, according to the publication, to determine the calcium concentration inside cells.

The disadvantage of the methods that have been described is that none of the above-described solutions are suitable for long-term use in the human or animal body. One possibility is even to use related sensor units in the body, as in the case of the variant that uses the fluorescence quenchers QSY-9, as discussed above, see Medintz et al.: In that case, molecules that have an unknown effect on the body can enter the body (the fluorescence quencher in this case). Moreover, the systems that are described are not stable in vivo, in particular those that are based on displacement reactions since the appropriate reagents must be constantly resupplied due to the exchange with bodily fluids. In addition, the fluorophores are not always adequately stable under the conditions that exist in the body.

A problem to be solved by the present invention is therefore that of providing a sensor unit for the qualitative and/or quantitative in vivo determination of an analyte that is functionally stable under in vivo conditions and, in particular, does not require any additional reagents that can easily enter the body and potentially stress it. The sensor unit should be capable of emitting optical signals, and their components should have high photostability and quantum yield. Furthermore, it is preferable to design the sensor unit to be as independent of pH value as possible.

### SUMMARY

According to the invention, the above problem, among others, is solved by a sensor unit that is functionally stable in the implant, for performing a qualitative and/or quantitative in vivo determination of an analyte, comprising:
(i) a connection area (3) having at least one binding site for the analyte (7), wherein the connection area undergoes a spatial change when the analyte binds to the binding site,
(ii) a first fluorescently active region (1), and
(iii) a second fluorescently active region (5/9),
wherein the first fluorescently active region (1) and the second fluorescently active region (5/9) are bound to the connection area (3) in a manner such that, when a spatial change occurs to the connection area (3) due to the analyte binding to the connection area (3), the distance between the first fluorescently active region (1) and the second fluorescently active region (5/9) changes without the bonds of the fluorescently active regions (1, 5/9) to the connection area being broken, and wherein one or both of the fluorescently active regions (1, 5/9) is comprised ≧60% by weight, and preferably ≧70% by weight of an anorganic material.

“Functionally stable in the implant” means, within the scope of the present invention, that the sensor unit is capable of generating at least up to 80% of the original signal intensity in the implanted state and upon potential contact with the particular analyte, given an identical concentration of the analyte, for a period of ≧30 days, preferably ≧60 days, further preferably ≧100 days, further preferably ≧150 days, and particularly preferably ≧one year. For a person skilled in the art, it goes without saying that an appropriate claim can be made with statistical significance only for a large number of (individual) sensor units. In the sense of the present description, the original signal intensity is the value that the sensor unit delivered immediately after implantation, for the given constellation.

Basically any molecule is feasible for use as the connection area that has the binding site for the analyte and undergoes a spatial change when the binding event occurs. One example of such a spatial change is, e.g., a conformity change of a protein. Thus, proteins are also preferred connection areas. Preferable molecules that selectively bind the analyte are glycoproteins, such as the glucose transporter of the bacterial strain E. coli or the human glucose receptor, or calmodulin or fragments or derivatives thereof, and molecularly designed or molecularly imprinted molecules. Moreover, the binding event can be facilitated by host-guest complexes, e.g., ion-selective crown ethers and cryptands, or by valinomycin or other ionophores.

A fluorescently active region, in the sense of the present invention, is a region that can induce fluorescence (fluorophore) or quench fluorescence (quencher). Within the scope of the invention, it goes without saying that at least one of the two fluorescently active regions must be a fluorophore. The fluorescently active regions to be used according to the invention are comprised of considerable portions of anorganic materials, which can positively influence their fluorescence properties and the stability. The expression “fluorescently active region” in the context of stating the percent by weight of anorganic material actually refers only to the region that is involved in the fluorescent result, i.e., the transmission and receiving of corresponding quanta. A possible coating of a “fluoroscent core” is explicitly not included in the calculation of the relative portion of anorganic material.

In the sense of the present definition, the fluorescently active region is preferably comprised of ≧85% by weight, further preferably ≧90% by weight, further preferably ≧95% by weight, and particularly preferably 100% by weight of anorganic material.

The detection system is based on the selective, specific, reversible binding of the analyte to a sensitive molecule (the sensor unit). Due to the change in the spatial structure of the sensitive molecule by the binding of the analyte, the distance between the at least partially anorganic fluorophore (first fluorescently active region) and a quencher or a second fluorophore, which is likewise at least partially anorganic (the second fluorescently active region) changes. According to the invention, they are both a fixed component of the sensor unit.

The fluorescence signal generated by the sensor unit according to the invention is modulated as a function of the binding event (the presence of one or more bonds of the analyte to the sensor unit, or the non-presence of such bonds). Preferred modulations in this context are, for example, distance-dependent fluoroscence quenching (quench) or fluorescence-resonance-energy-transfer (“FRET”), which cause a change in fluorescence intensity or the fluorescence duration of the anorganic fluorophore dependence on analyte concentrations. Optionally, a preferred modulation can also be a wavelength displacement of the light that is emitted during the fluoroscence event compared to the light that was received. The analyte concentration can be determined as a function of the identity and/or duration of the fluorescence signal using a calibration curve. Purely qualitative applications are also feasible, of course, since simply the presence or absence of a certain signal can be sufficient to qualitatively verify a binding event.

In conjunction with the present invention, it is preferable for the two fluorescently active regions to be separated from each other within the Förster radius before the binding event, and to be separated from each other beyond the particular Förster radius after the binding event. Depending on the design of the connection area, the exactly reversed case is also feasible, of course, namely that, before the binding event, the first fluorescently active region and the second fluorescently active region are separated from each other beyond the particular Förster radius, and are separated from each other within the Förster radius after the binding event. Thus, it is preferable for the radius of the fluorescently active regions relative to each other to be in the range of 1 to 10 nm, and preferably 2 to 6 nm for the state in which the (stronger) fluorescence signal is to be generated. For the state in which the weaker fluorescence signal or, preferably, no fluorescence signal should be generated, the distance between the two fluorescently active regions is ≧4 nm, preferably ≧7 nm, further preferably ≧10 nm, and particularly preferably 12 nm. The claims made in this paragraph apply for the situation in which FRET plays a decisive role in signal generation.

In the case of fluorescence quenching, the distances between the two fluorescently active regions is—for the case in which the (stronger) signal is to be generated—preferably ≧2 nm, further preferably ≧4 nm, and particularly preferably ≧6 nm; and for the case in which the weaker signal or no signal should be produced, it is ≦6 nm, preferably ≦4 nm, and particularly preferably ≦2 nm.

The advantage of the sensor unit according to the invention is, e.g., the stability thereof. By using selected anorganic materials for at least one fluorophore (see further below), high photostability and quantum yield can be attained, as well as a low pH-value dependence of the fluorescence that can be generated by the sensor according to the invention. Surprisingly, this overcomes the previous stability problem of fluorescence-based detection systems that are designed to deliver in vivo measured results when implanted. At the same time, it is not necessary to use additional reagents that influence the fluorescence properties (e.g., quenchers that are reversibly bindable to the sensor). Basically, the sensor unit can therefore be left in the body for a long period of time without exogenous compounds acting on it. In addition, the sensor unit does not require any reagents that could potentially be harmful to the body and that are only reversibly bound.

A sensor unit according to the invention is preferred, in the case of which one or both of the fluorescently active regions may be a coated, fluorescently active nanoparticle.

In this context, it is furthermore preferable for the nanoparticle to be selected from the group comprised of quantum dot, nanophosphor, and dye-labeled silicate.

In the sense of the present invention, nanoparticles are particles having a size of 1 nm to 200 nm, relative in each case to the largest diameter of the particle, preferably in the range of 3 to 100 nm. The particle size is preferably determined using light scattering according to, for example, ISO 22412.

In particular, quantum dots according to the present invention are understood to be fluorescent anorganic nanoparticles that are semiconducting materials, are comprised of II-VI- or III-V-semiconductors, and preferably have a nuclear membrane structure. Semiconducting materials of that type preferably comprise an anorganic core that is preferably less than 10 nm in size (the largest diameter is noted above). Preferably, such semiconducting nanoparticles are capped, i.e., they are enclosed in an organic shell, for example. To solubilize the nanoparticles in the aqueous medium, it is preferable to modify the surface of the nanoparticle structures with functional protective layers without changing the optical properties. This includes modifications with, for example, thiol monolayers, thiolated siloxanes, glutathiones, peptides, proteins, dendrites, polyethylene glycol, monosaccharides, disaccharides, trisaccharides, low-molecular polysaccharides, hydrophilic vitamins, lipophilic vitamins, fatty acids, polyalcohols, Teflon, amino acids, non-specific peptides or proteins, phosphorylcholine, polylactate, and/or derivatives of the aforementioned compounds. Another possibility is the binding of long hydrophobic chains that form bilayers in combination with phospholipids or diblock copolymers. Alternative possibilities include, for example, coating the particles with silica, dendrimers, amphiphilic polysaccharides and polymers. The connection area (the sensitive molecule) may also be bound to these surface modifications using physical adsorption or, preferably, covalently, by a linker.

Preferred quantum dots in the present invention are semiconducting materials selected from the group comprised of InAs, InP, GaAs, GaP, GaN, InGaAs, GaInP/InP, CdO, CdSe, CdS5CdTe ZnO, ZnS, ZnSe, ZnTe, PbS, PbSe, PbTe, HgS, HgSe and HgTe, and particularly preferably CdSe, CdS or CdTe.

In the present application, nanophosphors are understood to be anorganic nanoparticles that are comprised of non-conductive or semiconducting materials and are doped with rare earth ions. They are thus preferably semiconducting materials. In principle, the following compounds can be selected as the material for the doped nanophosphors, wherein, in the text below, one or more doping elements are listed to the right of the colon, and the host material is stated to the left of the colon. The host material can be compounds from the group of, for example, phosphates, silicates, germanates, oxides, sulphides, oxysulphides, selenides, sulphoselenides, vanadates, niobates, arsenates, tantalates, wolframates, molybdates, halogenates, nitrides, borates, aluminates, gallates, and halogenides. Particularly preferred are nanophosphors manufactured on the basis of lanthanum phosphates and/or yytrium-vanadium oxide. Examples are LiLEu; Al₂O₃:Eu; BaFCl:Sm; BaFBnEu; BaY₂F₈:Ln (Ln-Pr, Tm, Er, Ce), BaMgAl₁₆O₂₇═Eu; BaMgAl₁₄O₂₃:Eu; BaMgAl₁₀O₎₇:Eu; BaMgAl₂O₄═Eu₁Ce(Mg₁Ba)Al₁₁Oi₉; MgAl₁₁O₁₉:Ce; (Mg,Ca)S:Eu; MgWO₄:Sm; CaS:Ln (Ln=lanthanoids); CaWO₄;Sm; CaSO₄:Ln (Ln=lanthanoids); SrS:Ln (Ln=lanthanoids), Sr₂P₂O₇; SrGa₂S₄:Ln (Ln=lanthanoids); YF₃:Ln (Ln=lanthanoids); Y₂O:Ln (Ln=lanthanoids); Y(P,V)O₄:Eu; YOCl:Yb,Er; LuVO₄:Eu; GdVO₄:Eu; Gd₂O₂SjTb; GdMgB₅O₁₀:Ce₅Tb; LaOBr:Tb; La₂O₂S:Tb; LaF₃:Nd,Ce; BaYb₂F₈:Eu; NaYF₄:Yb₅Er; NaGd F₄:Yb₁Er; NaLaF₄:Yb₅Er; LaF₃:Yb₁Er₅Tm; BaYF₅:Yb,Er; Ga₂O₃:Dy; GaN:A (A=Pr, Tm₅ Er, Ce); Gd₃Ga₅Oi₂=Tb; LiLuF₄:A (A-Pr₅ Tm₅ Er, Ce); CaSiO₃:Ln; CaS:Ln; CaO:Ln; ZnS:Ln; MgF₂:Ln with Ln=lanthanoids YVO₄:Ln; LnPO₄:Ce,Tb; Y₂O₃:Ln; Y₂O₂S:Ln; Y₂SiO₅:Ln with Ln=lanthanoids.

Preferably, the nanophosphors have an anorganic core which is enclosed by an organic shell. Moreover, it is preferable for nanophosphors to have an anorganic core, the maximum diameter of which is preferably less than 10 nm.

The emission spectrum depends on the doping that is used, and not on diameter, as is the case with quantum dots. In synthesis, the size of the nanophosphors does not have to be adjusted within such narrow limits as for quantum dots. The advantage of nanophosphors is likewise the photostability.

As an alternative, silica nanoparticles filled with dyes can be used; they are referred to as dye-doped silica nanoparticles. They do not bleach out, can be used easily in the aqueous medium due to the outer layer, and facilitate easy coupling of the connection area.

As described above, it can be preferably for one of the fluorescently active regions to be a quencher. Quenchers that are preferred in the context of the present invention are, for example, nanoparticles of metals such as gold and silver, and fullerenes. Organic quenchers are likewise preferred, such as, for example, Doxyl, members of the DDQ family, dabcyl, Eclipse, members of the Iowa Black family, members of the BHQ family, members of the QSY family, heterocyclic quinone derivatives, and DABCYL. These quenchers have proven particularly effective in the sense of the invention.

In the sense of the invention, a sensor unit according to the invention is preferred, in the case of which the analyte is selected from the group comprised of electrolytes, carbohydrates, metabolites, metabolic products, amino acids, peptides, fats, fatty acids, lipids, proteins, neurotransmitters, polyelectrolytes, ribonucleic acids, deoxyribonucleic acids, nucleotides, hormones, and active agents.

The following analytes can be preferable, in particular: Albumins/globulins, alkaline phosphatase, alpha-1-globulin, alpha-2-globulin, alpha-1-antitrypsin, alpha-1-fetoprotein, alpha-amylases, alpha-hydroxybutyrate-dehydrogenase, ammonia, antithrombin III, bicarbonate, bilirubin, erythrocyte sedimentation rate, bleeding time, carbohydrate antigen 19-9, carcinoembryonic antigen, chloride, cholesterol, cholinesterase, cobalamin/vitamin B12, coeruloplasmin, C-reactive protein, cystatin C, D-dimers, iron, erythropoetin, erythrocytes, ferritin, fetuin-A fibrinogen, folic acid/vitamin B9, free tetrajodthyronine (fT4), free trijodthyronine (fT3), gamma-glutamyl transferase, glucose, glutamate dehydrogenase, glutamate oxaloacetate transaminase, glutamate pyruvate transaminase, glycohemoglobin, hematocrit, hemoglobin, haptoglobin, uric acid, urea, HDL cholesterol, homocysteine, immunoglobulin A, immunoglobulin E, immunoglobulin G, immunoglobulin M, INR, calium, calcium, creatinine, creatine kinase, copper, lactate, lactate dehydrogenase, LDL cholesterol, leukocytes, lipase, lipoprotein, magnesium, mean corpuscular hemoglobin concentration, mean corpuscular hemoglobin, mean corpuscular volume, myoglobin, sodium, NT-proBNP/BNP, osmolality, phosphate, pH value, plasma-thrombin coagulation time, prostate-specific antigen, reticulocytes, rheumatoid factor, thrombocytes, thyroid stimulating hormone, transferrin, triglycerides, troponin T, muscarinic receptor antagonists, neuromuscular blocking substances, cholesterol esterase inhibitors, adrenoceptor agonists, indirectly acting sympathomimetics, methylxanthine, alpha-adrenoreceptor antagonists, ergot alkaloids, beta-adrenoceptor antagonists, inactivation inhibitors, antisympathonics, 5-HT receptor agonists, histamine receptor agonists, histamine receptor antagonists, analgesics, local anesthetics, sedatives, anticonvulsants, convulsants, muscle relaxants, antiparkinsonians, neuroleptics, antidepressants, lithium, tranquilizers, immunosuppressants, antirheumatics, antiarrhythmics, antibiotics, ACE inhibitors, aldosterone receptor antagonists, diuretics, vasodilatators, positive inotropic substances, antithrombotic/thrombolytic substances, laxatives, antidiarrheal agents, pharmaceuticals for adiposity, uricostatics, uricosurics, antilipemics, antidiabetics, antithypoglycemia, hormones, iodized salts, threostatics, iron, vitamins, trace elements, virostatics, antimycotics, antituberculotics, substances for tumor chemotherapy, and their derivatives.

The sensor unit according to the invention is particularly well suited for use to detect the aforementioned analytes, since they are regularly present in the bodily fluids. The sensor unit according to the invention is basically particularly well suited for use in bodily fluids or at least in contact with bodily fluids, since the fluids ensures that the particular analyte will be easily transported and/or diffused to the sensor unit.

In the context of the invention, the analyte is preferably glucose or calcium.

Basically, a person skilled in the art may be flexible in terms of selecting the design of the sensor unit. The decisive factor is that the first fluorescently active region and the second fluorescently active region are bound to the connection area in a manner such that it undergoes a spatial change when the analyte is bound to a binding site thereof; the spatial change, in turn, influences the distance between the fluorescently active regions. As described above, the binding of the fluorescently active regions can take place, e.g., using a linker, or can take place directly to the C-terminus and/or N-terminus of the connection area (e.g., if this is a protein); it can likewise be provided on suitable side chains of the connection area.

An implant comprising a sensor unit according to the invention is also a component of the invention. This implant regularly comprises an outer region (possibly outer walls and/or membrane) which limits the contact of the sensor unit according to the invention with the physical environment. An implant according to the invention preferably comprises a semipermeable membrane, which is permeable to the analyte, of course, in the area of the outer region.

By installing the sensor unit within the framework of an implant in the body, it is possible to reduce or even prevent contact between physical components (and components of bodily fluids) and the sensor unit. The functional stability of the sensor unit can be ensured in this manner. This takes place, e.g., in that proteases are prevented from accessing the sensor unit.

Furthermore, a suitable outer shell (possibly including a semipermeable membrane) can prevent competing molecules, or molecules that disturb the detection system, from coming into contact with the sensor unit according to the invention.

It is furthermore preferred according to the invention for the sensor unit according to the invention to be immobilized inside the implant. This can take place using, for example, permanent bonding to the interior of the outer shell of the implant, or by bonding to a matrix inside the implant. It is preferred according to the invention for the sensor unit according to the invention to be immobilized inside the implant with a polymer, preferably hydrogel. The immobilization results in a defined position of the sensor unit within the implant, and possibly to stabilization of the sensor unit according to the invention. As a further advantage, the sensor unit cannot enter the body in an uncontrolled manner even if the outer shell of the implant becomes damaged. If a hydrogel was used for the immobilization, this has the advantage that it is permeable to small analytes, in particular, thereby ensuring that these analytes will easily come in contact with the sensor unit according to the invention.

If the sensor unit is immobilized in the implant of a matrix, then it goes without saying that the matrix is designed such that it is permeable to the applicable fluorescent light. As an alternative, it is also possible to situate the matrix such that it is not located between the sensor unit and an appropriate fluorescence detector.

In the sense of the present invention, the semipermeable membrane can be constructed of various layers, each of which performs certain functions. A stabilizing membrane part preferably prevents larger objects, such as, for example, cells from entering the interior of the implant, and preferably ensures that the sensor units and the matrix remain in the interior. An additional membrane layer ensures that the particles and molecules that disrupt and interfere with the detection system are kept away from the interior of the implant. An angiogenic layer can cause new blood vessels to form in the vicinity of the implant. The surface of the semipermeable membrane can have hydrophobic or hydrophilic properties, depending on the application, and they can have a cationic or anionic or metallic character. To prevent biofouling, a number of modifications can be applied to the membrane covalently or via physical adsorption. Anorganic or organic molecules can be bonded to the surface via physical adsorption or covalent bonds, such as, for example, polymers, peptides, proteins, aptamers, molecularly imprinted polymers, RNA, DNA, siRNA, and nanoparticles. Depending on the application, a typical diameter of the pores of the membranes (typical thickness of 0.5-10 μm) feasibly has values in the range 1 nm to 10 μm, thereby allowing the analyte to be detected to pass through the membrane, but preventing particles or molecules that cause interference or disrupt the detection system from passing through the membrane.

As an alternative, a partially bioresorbable membrane is also feasible.

In the sense of the invention, an implant according to the invention is preferred that comprises surface designs or modifications on the outer walls of the implant, which improve the biocompatibility of the implant, control the adhesion behavior, and/or reduce thrombogenesis.

These surface modifications can apply to the entire shell of the implant, even including a membrane, provided the particular surface modification does not eliminate the permeability of the membrane to the analytes. The surface functionalization of the implant or parts of the implant can be used to improve the body's reaction to the implant. This includes, for example, the adhesion behavior, inflammatory responses, the “foreign body response”, and the prevention of biofouling.

The surface of the implant (or a portion of the surface) can have hydrophobic or hydrophilic properties, depending on the application. It can have a cationic or anionic or metallic character. Anorganic or organic molecules can be bonded to the surface via physical adsorption or covalent bonds such as, for example, polymers, peptides, proteins, aptamers, molecularly imprinted polymers, RNA, DNA, siRNA, and nanoparticles. The surface can have nanostructuring or microstructuring. To provide the structure, round, spherical, cylindrical, conical, square, rectangular, or elongated structures can be applied to or removed from the surface. They can include, for example, grooves, tubes, solid cylinders, balls, hemispheres, cuboids, and cubes.

A partially bioresorbable surface is also feasible.

Depending on where the implant is planned to be placed in the body, all of these embodiments can be used to improve the aforementioned responses, in particular, in the sense of improved biocompatibility.

The advantage of a partially bioresorbable implant (in terms of the outer wall thereof) is that the bioresorption creates microconditions in the region of the implant that help prevent unwanted physical responses and promote desired physical responses (such as, for example, adhesion).

An implant is preferred according to the invention that comprises a fluorescence detector and a device for transmitting the values measured by the detector out of the implant.

Such a detector can be, e.g., a photodiode, photoresistor, phototransistor, photomultiplier, or a pyroelectrical sensor. The device used to transmit the values measured using the detector can utilize technologies such as, for example, surface acoustic waves (“SAWs”), inductive coupling, and RF telemetry.

The invention also relates to the use of a sensor unit according to the invention, or an implant according to the invention to perform the qualitative or quantitative in vitro determination of an analyte.

This potential use is a particular advantage of the sensor unit according to the invention or the implant according to the invention. Due to their design, they are capable of determining data of an analyte in vitro for a relatively long period of time. The following statements apply for the transmission and evaluation of data.

The invention also relates to a method for performing the qualitative or quantitative in vivo determination of an analyte, comprising the steps of:
a) bringing a sensor unit of the type described above or an implant of the type described above in contact, in or on the body, with a bodily fluid of a human or an animal that at least potentially contains the analyte,
b) registering the fluorescence signal generated by the sensor unit, and
c) comparing the signal to a reference value or an array of reference values to qualitatively or quantitatively determine the analyte.

It is basically possible to allow step b) of the method according to the invention to take place in the body of the human or the animal, or outside of the body of the human or the animal.

It is decisive, of course, that the sensor unit/implant is applied in or on the body in a manner such that the analyte can potentially come in contact with the sensor unit. This can take place, e.g., by bringing the implant in contact with the bloodstream or the bodily fluid, thereby enabling the analyte to come in contact with the sensor unit (or possibly even via a membrane of the implant). If such contact does occur, the fluorescence signal emitted by the sensor unit changes. As indicated above, these fluorescence signal can be, e.g., the intensity (frequency domain) of the signal or the course (time domain).

As indicated above, two basic cases exist:

### Case 1) The Analytical System is Located Outside of the Body.

Using a light source (e.g., a laser) and a detector (photodiode), which have been adapted to the optical properties of the detection system (e.g., wavelength, output, etc.), the optical properties of the detection system are determined as a function of the analyte concentration. To do this, the first fluorescently active region is excited. The fluorescence of the donor, the acceptor, or the donor and the acceptor, or the fluorescence duration of the donor can be determined and evaluated, thereby enabling the concentration of the analyte in the measurement solution to be deduced. The implant thus contains no electronic components.

### Case 2) The Analytical System is Located Inside the Implant.

Analogous to case 1), the optical properties are likewise determined as a function of the analyte concentration (fluorescence of the donor, of the acceptor, of the donor and the acceptor, or the fluorescence duration of the donor). These can be evaluated using a suitable electronic system in the implant and transmitted via telemetry to an external receiver. The power supply (battery) provides the energy required for detection and data transmission. An embodiment is also feasible in which energy is supplied from the outside.

The measured values that are obtained in both cases are evaluated by comparing the particular signal with a calibration curve. This comparison can also take place automatically, of course. The calibration curve must be determined in a manner such that signals are generated as a function of different concentrations of the particular analyte under conditions that are comparable to the conditions for use. This is feasible, e.g., for the case of an implanted glucose concentration measuring unit, in that signals are generated as a function of different glucose concentrations, and these signals are directed through skin in vitro, in which case the thickness of the skin corresponds to the implantation depth. Measures of glucose concentration are also generated in a comparable system (e.g., blood/interstitial fluid), of course.

By performing a comparison with the appropriate calibration curve, it is therefore possible to deduce the physiological state of the human/animal directly, possibly in real time. The calibration curve is determined by analyzing a plurality of reference solutions that contain the analyte in the physiologically relevant range and determining the corresponding measurement signal (fluorescence or fluorescence duration).

Action can then be taken depending on the particular in vitro evaluation result. It is possible, e.g., in the case of hyperglycemia, to trigger an (increased) dosage of insulin using an insulin pump which may be connected. In the case of hypoglycemica, however, the administration of insulin could be throttled. The morbidity and mortality of patients with diabetes mellitus can be reduced in this manner.

The same applies for the response to the data measured on other analytes such as, for example, calcium and clinical pictures coupled to this analyte.

A further advantage of the implant according to the invention is that a high stability of the sensor unit can be obtained by using a suitable design. It is therefore possible to manufacture sensor units in the sense of a durable system for determining the concentration of the particular analyte, and to implement them.

Various other objects, aspects and advantages of the present invention can be obtained from a study of the specification, the drawings, and the appended claims.

### DESCRIPTION OF THE DRAWINGS

FIG. 1 shows a sensor unit according to the invention, the measurement signal of which is influenced by FRET.

FIG. 2 shows a sensor unit according to the invention, the measurement signal of which is influenced by quench.

It should be pointed out once more that, in general, the measurement signal can be, e.g., the intensity or duration of the fluorescence.

### DETAILED DESCRIPTION

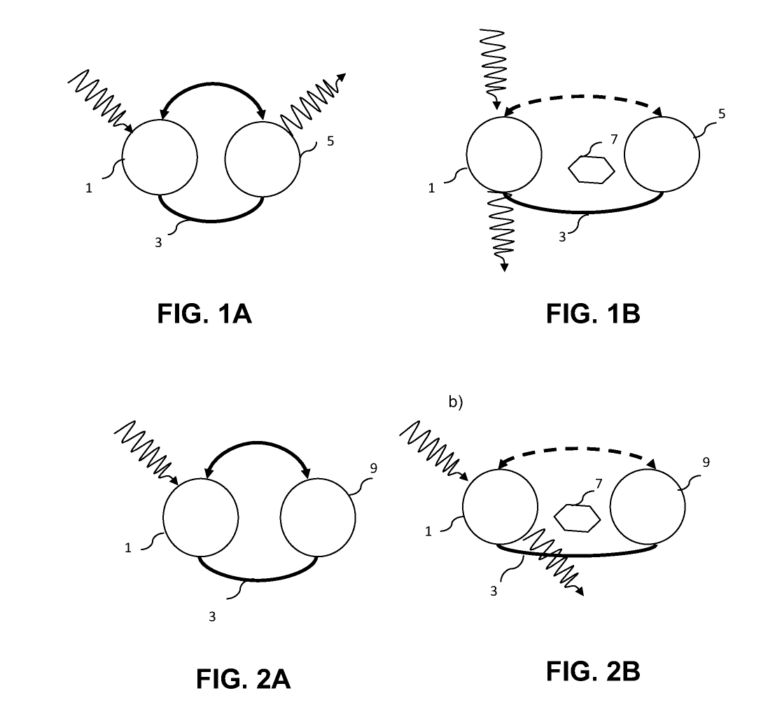

FIG. 1A shows the situation in which, without analyte 7 on connection area 3, the first fluorescently active region 1 (fluorophore) and the second fluorescently active region 5 (fluorophore) are spatially close to each other and, therefore, a fluorescence resonance energy transfer (“FRET”) takes place. The binding event of analyte 7 to connection area 3 (FIG. 1B) causes the distance between the first fluorescently active region 1 and the second fluorescently active region 5 to increase and, therefore, FRET does not occur, or occurs only to a reduced extent. The fluorescence signal therefore changes.

In FIG. 2, in the case of FIG. 2A, the first fluorescently active region 1 (fluorophore) is located in the spatial vicinity of the second fluorescently active region 9 (quencher) and, therefore, at least a portion of the fluorescence is quenched. In FIG. 2B, the spatial distance between the two fluorescently active regions 1 and 9 has been increased by the binding event of analyte 7 to connection area 3. As a result, the fluorescence quenching (quench) is reduced, and the fluorescence signal changes as a function of the binding result.

Example using a Glucose Sensor:

To perform an optical determination of the concentration of glucose in bodily fluids, a fluorescent nanoparticle (nanophosphor, YAG:Ce3+) is bound to the glucose binding protein (GBP) of the E. coli strain, on the N terminus, and a fluorescence quencher (gold nanoparticle) is bound to another position which is spatially separated from the first position. The distance from the fluorescent nanoparticle to the quencher is approximately 4 nm. As an alternative to the quencher, a further fluorescent nanoparticle (nanophosphor or quantum dot) can also be applied.

For binding to the sensitive molecule (connection area, GBP in this case), the surface of the fluorescent nanoparticle is functionalized by using APTMS (3-aminopropyltrimethoxysilane) and 1,4-BD (1,4-butanediol) in a manner such that it is provided with a biotin function via the addition of SB (sulfosuccinimidyl-6-(biotinamido)-hexanoate biotinylation agent). The GBP is provided, at the N terminus, with a monovalent streptavidin (has only one biotin binding pocket) via a biotechnological modification (recombinant). The optical properties are not changed as a result. This procedure is based on Azakura et al., Tagging of Avidin Immobilized Beads With Biotinylated YAG:Ce3+ Nanocrystal Phosphor, Anal Bioanal Chem (2006) 386:1641-1647.

The fluorescence quencher (gold nanoparticle) is connected to the C terminus of the GBP as follows: A cysteine tag composed of 5 cysteines is attached to the C terminus (together with the recombinant modification of the N terminus). The gold nanoparticles are incubated with a mixture of mPEG-SH and HS-PEG-NH2 (PEG=poly(ethylene glycol)). After addition of the crosslinker (4-(N-maleimidomethyl)cyclohexane-1-carboxylic-acid-3-sulfo-N-hydroxysuccinimide-ester-sodium salt), the nanoparticles are covalently bound to the N-terminal thiol groups of the GBP. This procedure is based on Li et al., Immobilization of Glucose Oxidase Onto Gold Nanoparticles With Enhanced Thermostability, Biochemical and Biophysical Research Communications 355 (2007) 488-493. Although, in deviation there from, a cysteine tag with n≧1 has proven particularly effective in the context of the invention.

The entire molecule is created by combining the streptavidin-GBP-gold nanoparticle with the biotinylated fluorescent nanoparticle.

### Mode of Operation of the Sensor:

### Case 1)

In the glucose-free form, GBP has a relatively compact structure; i.e., the distance between the fluorescent nanoparticle and the fluorescence quencher is small (e.g., the distance is approximately 3 nm). The fluorescence is therefore quenched. Now, if more and more glucose molecules bind to the glucose receptor, the receptors undergo a conformational change which is observed as an increase in the overall fluorescence (reduced quench). The values for the fluorescence duration have changed.

### Case 2)

In the case of the above-described alternative, in an analogous manner, two fluorescent nanoparticles (donor and acceptor (see above)) can be attached to the glucose receptor (connection area). In the glucose-free state, the two fluorescent particles are located relatively close to each other (e.g., the distance is approximately 6 nm). As a result, the energy can be transmitted without radiation from the donor to the acceptor using resonance energy transfer. The fluorescence of the acceptor is great. If glucose binds to the GBP, the distance between the donor and the acceptor increases, the fluorescence of the acceptor is reduced, and the values for the fluorescence duration have changed.

For use in vivo, the concentration of the above-described sensor units is selected such that not all sensors are saturated at physiologically relevant concentrations of glucose. Typical glucose concentrations are 0.1 to 25 mM glucose.

In the present example, the glucose concentration is determined via comparison with a calibration curve. For this purpose, the associated fluorescence intensity and/or fluorescence duration are determined as a function of glucose concentration using reference solutions (e.g., 0, 5, 10, 15, 20, 25 mM glucose) in the physiologically relevant range of, e.g., 0-25 mM glucose.

By comparing the particular measurement value against the calibration curve, it is therefore possible to determine the in situ concentration of the analyte using the sensor unit presented as an example (or using a large number of these sensor units).

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

### REFERENCE NUMERALS

1 First fluorescently active region (fluorophore)

3 Connection area

5 Second fluorescently active region (fluorophore)

7 Analyte

9 Second fluorescently active region (quencher)

I/we claim:

1. A sensor unit that is functionally stable in an implant, for performing a qualitative and/or quantitative in vivo determination of an analyte, the sensor unit comprising:
(i) a connection area having at least one binding site for the analyte, wherein the connection area undergoes a spatial change when the analyte binds to the at least one binding site;
(ii) a first fluorescently active region; and
(iii) a second fluorescently active region, wherein the first fluorescently active region and the second fluorescently active region are bound to the connection area in a manner such that, when a spatial change occurs to the connection area due to the analyte binding to the connection area, the distance between the first fluorescently active region and the second fluorescently active region changes without the bonds of the fluorescently active regions to the connection area being broken, and wherein one or both of the fluorescently active regions is comprised of ≧60% by weight of an anorganic material.

2. The sensor unit according to claim 1, wherein one or both of the fluorescently active regions is a coated, fluorescently active nanoparticle.

3. The sensor unit according to claim 2, wherein the nanoparticle is selected from the group consisting of quantum dot, nanophosphor, and dye-labeled silicate.

4. The sensor unit according to claim 1, wherein the analyte is selected from the group consisting of electrolytes, carbohydrates, metabolites, metabolic products, amino acids, peptides, fats, fatty acids, lipids, proteins, neurotransmitters, polyelectrolytes, ribonucleic acids, deoxyribonucleic acids, nucleotides, hormones, and active agents.

5. The sensor unit according to claim 1, wherein the analyte is glucose, calium, calcium, a peptide, or a protein.

6. An implant comprising a sensor unit according to claim 1.

7. The implant according to claim 6, further comprising a membrane that is permeable to the analyte.

8. The implant according to claim 6, wherein the sensor unit is immobilized inside the implant.

9. The implant according to claim 8, wherein the sensor unit is immobilized in a matrix or on interior walls of the implant.

10. The implant according to claim 6, further comprising a fluorescence detector and a device for transmitting the values measured by the fluorescence detector out of the implant.

11. The implant according to claim 6, further comprising surface designs or modifications on outer walls of the implant, which improve the biocompatibility of the implant, control the adhesion behavior, and/or reduce thrombogenesis.

12. The implant according to claim 11, wherein a portion of the outer wall of the implant is bioresorbable.

13. Use of a sensor unit according to claim 1, for the qualitative or quantitative in vivo determination of an analyte.

14. Use of an implant according to claim 6, for the qualitative or quantitative in vivo determination of an analyte.

15. A method for performing the qualitative or quantitative in vivo determination of an analyte, comprising the steps of:
a) bringing a sensor unit according to claim 1 in contact, in or on the body, with a bodily fluid of a human or an animal that at least potentially contains the analyte;
b) registering the fluoroscence signal generated by the sensor unit; and
c) comparing the generated fluoroscence signal to a reference value or an array of reference values to qualitatively or quantitatively determine the analyte.

16. The method according to claim 15, wherein step b) takes place (i) in the body of the human or the animal, or (ii) outside of the body of the human or the animal.

17. A method for performing the qualitative or quantitative in vivo determination of an analyte, comprising the steps of:
a) bringing an implant according to claim 6 in contact, in or on the body, with a bodily fluid of a human or an animal that at least potentially contains the analyte;
b) registering the fluoroscence signal generated by the sensor unit; and
c) comparing the generated fluoroscence signal to a reference value or an array of reference values to qualitatively or quantitatively determine the analyte.

18. The method according to claim 17, wherein step b) takes place (i) in the body of the human or the animal, or (ii) outside of the body of the human or the animal.
